(19) United States
(12) Patent Application Publication
(10) Pub. No.: US 2024/0278004 A1
(43) Pub. Date: Aug. 22, 2024

(54) IMPLANTABLE MEDICAL SYSTEMS FOR CANCER TREATMENT WITH ELECTRODES FOR THERMAL MANAGEMENT

(71) Applicant: Cardiac Pacemakers, Inc., St. Paul, MN (US)

(72) Inventors: Devon N. Arnholt, Shoreview, MN (US); Michael J. Kane, St. Paul, MN (US); Brian L. Schmidt, White Bear Lake, MN (US)

(73) Assignee: MAYO FOUNDATION FOR MEDICAL EDUCATION AND RESEARCH, Rochester, MN (US)

(21) Appl. No.: 18/443,066

(22) Filed: Feb. 15, 2024

Related U.S. Application Data
(60) Provisional application No. 63/446,411, filed on Feb. 17, 2023.

(51) Int. Cl. A61N 1/05

(52) U.S. Cl. CPC A61N 1/0529

(57) ABSTRACT

Embodiments herein relate to implantable systems for cancer treatment including electrodes that can aid in thermal management. In an embodiment, an implantable lead for a cancer treatment system is included having a lead body with a proximal end and a distal end. The lead body can define sides of a tissue exclusion channel. The lead can include one or more supply electrodes, wherein the electrodes are disposed along a length of the lead body. The tissue exclusion channel can be disposed circumferentially around the electrodes. Other embodiments are also included herein.

This application claims the benefit of U.S. Provisional Application No. 63/446,411, filed Feb. 17, 2023, the content of which is herein incorporated by reference in its entirety.

### FIELD

Embodiments herein relate to implantable systems for cancer treatment and related methods. More specifically, embodiments herein relate to implantable systems for cancer treatment including electrodes that can aid in thermal management.

### BACKGROUND

According to the American Cancer Society, cancer accounts for nearly 25% of the deaths that occur in the United States each year. The current standard of care for cancerous tumors can include first-line therapies such as surgery, radiation therapy, and chemotherapy. Additional second-line therapies can include radioactive seeding, cryotherapy, hormone or biologics therapy, ablation, and the like. Combinations of first-line therapies and second-line therapies can also be a benefit to patients if one particular therapy on its own is not effective.

Cancerous tumors can form if one normal cell in any part of the body mutates and then begins to grow and multiply too much and too quickly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Various cancer therapies may have significant side effects on heathy tissue. Such side effects can vary widely depending on the type of cancer therapy but can manifest as anemia, thrombocytopenia, edema, alopecia, infections, neutropenia, lymphedema, cognitive problems, nausea and vomiting, neuropathy, skin and nail changes, sleep problems, urinary and bladder problems, and the like.

### SUMMARY

Embodiments herein relate to implantable systems for cancer treatment and related methods. In a first aspect, an implantable lead for a cancer treatment system can be included having a lead body. The lead body can include a proximal end and a distal end. The lead body can define sides of a tissue exclusion channel. The lead can further include one or more supply electrodes, wherein the electrodes can be disposed along a length of the lead body. The tissue exclusion channel can be disposed circumferentially around the electrodes.

In a second aspect, in addition to one or more of the preceding or following aspects, or in the alternative to some aspects, the tissue exclusion channel can have a radial depth of 0.1 to 4 mm.

In a third aspect, in addition to one or more of the preceding or following aspects, or in the alternative to some aspects, the tissue exclusion channel can have an axial length of 1 to 200 mm.

In a fourth aspect, in addition to one or more of the preceding or following aspects, or in the alternative to some aspects, the implantable lead can further include one or more support flanges, wherein the one or more support flanges can be disposed within the tissue exclusion channel.

In a fifth aspect, in addition to one or more of the preceding or following aspects, or in the alternative to some aspects, the implantable lead can further include a rapidly dissolving composition, wherein the rapidly dissolving composition can be packed within the tissue exclusion channel.

In a sixth aspect, in addition to one or more of the preceding or following aspects, or in the alternative to some aspects, the electrodes can be disposed circumferentially around the lead body and extend a distance along a longitudinal axis of the lead body.

In a seventh aspect, in addition to one or more of the preceding or following aspects, or in the alternative to some aspects, the electrodes can include a flat ribbon of metal.

In an eighth aspect, in addition to one or more of the preceding or following aspects, or in the alternative to some aspects, the flat ribbon of metal can be wrapped around the lead body.

In a ninth aspect, in addition to one or more of the preceding or following aspects, or in the alternative to some aspects, the electrodes can include at least two supply electrodes.

In a tenth aspect, in addition to one or more of the preceding or following aspects, or in the alternative to some aspects, the at least two supply electrodes can be separated by a distance along a longitudinal axis of the lead body.

In an eleventh aspect, in addition to one or more of the preceding or following aspects, or in the alternative to some aspects, the implantable lead can further include a temperature sensor, wherein the temperature sensor can be disposed between the at least two supply electrodes.

In a twelfth aspect, in addition to one or more of the preceding or following aspects, or in the alternative to some aspects, the implantable lead can further include a coating, wherein the coating can be disposed over a surface of the supply electrodes, and wherein the coating can be formed of an expandable material.

In a thirteenth aspect, in addition to one or more of the preceding or following aspects, or in the alternative to some aspects, the implantable lead can further include a coating, wherein the coating can be disposed on the implantable lead at a location bordering the tissue exclusion channel, and wherein the coating can be formed of an expandable material.

In a fourteenth aspect, an implantable lead for a cancer treatment system can be included having a lead body with a proximal end and a distal end and one or more supply electrodes, wherein the electrodes can be disposed along a length of the lead body. A spacing ridge can be included, wherein the spacing ridge can be disposed on at least one of the lead body or the electrodes. The spacing ridge can extend radially from the implantable lead for a cancer treatment system and at least partially defines a tissue exclusion space adjacent the spacing ridge.

In a fifteenth aspect, in addition to one or more of the preceding or following aspects, or in the alternative to some aspects, the implantable lead can further include a second spacing ridge, wherein the spacing ridge and the second spacing ridge can be disposed on opposite sides of the electrodes.

In a sixteenth aspect, in addition to one or more of the preceding or following aspects, or in the alternative to some aspects, the electrodes can be disposed circumferentially around the lead body and extend a distance along a longitudinal axis of the lead body.

In a seventeenth aspect, in addition to one or more of the preceding or following aspects, or in the alternative to some aspects, the electrodes can include a flat ribbon of metal.

In an eighteenth aspect, in addition to one or more of the preceding or following aspects, or in the alternative to some aspects, the flat ribbon of metal can be wrapped around the lead body.

In a nineteenth aspect, in addition to one or more of the preceding or following aspects, or in the alternative to some aspects, the electrodes can include at least two supply electrodes.

In a twentieth aspect, in addition to one or more of the preceding or following aspects, or in the alternative to some aspects, the at least two supply electrodes can be separated by a distance along a longitudinal axis of the lead body.

In a twenty-first aspect, in addition to one or more of the preceding or following aspects, or in the alternative to some aspects, the implantable lead can further include a temperature sensor, wherein the temperature sensor can be disposed between the at least two supply electrodes.

In a twenty-second aspect, in addition to one or more of the preceding or following aspects, or in the alternative to some aspects, the spacing ridge can be formed of an expandable material.

In a twenty-third aspect, an implantable lead for a cancer treatment system can be included having a lead body including a proximal end and a distal end and one or more supply electrodes. The electrodes can include a proximal edge, wherein the proximal edge can be tapered inward forming a proximal tapered portion. The electrodes can also include a distal edge, wherein the distal edge can be tapered inward forming a distal tapered portion. The electrodes can be disposed along a length of the lead body.

In a twenty-fourth aspect, in addition to one or more of the preceding or following aspects, or in the alternative to some aspects, a diameter at the proximal edge can be at least 10% less than a diameter at a midpoint between the proximal edge and the distal edge.

In a twenty-fifth aspect, in addition to one or more of the preceding or following aspects, or in the alternative to some aspects, a diameter at the distal edge can be at least 10% less than a diameter at a midpoint between the proximal edge and the distal edge.

In a twenty-sixth aspect, in addition to one or more of the preceding or following aspects, or in the alternative to some aspects, the proximal tapered portion can be curved, and wherein the distal tapered portion can be curved.

In a twenty-seventh aspect, in addition to one or more of the preceding or following aspects, or in the alternative to some aspects, the electrodes can be disposed circumferentially around the lead body and extend a distance along a longitudinal axis of the lead body.

In a twenty-eighth aspect, in addition to one or more of the preceding or following aspects, or in the alternative to some aspects, the electrodes can include a flat ribbon of metal.

In a twenty-ninth aspect, in addition to one or more of the preceding or following aspects, or in the alternative to some aspects, the flat ribbon of metal can be wrapped around the lead body.

In a thirtieth aspect, in addition to one or more of the preceding or following aspects, or in the alternative to some aspects, the electrodes can include at least two supply electrodes.

In a thirty-first aspect, in addition to one or more of the preceding or following aspects, or in the alternative to some aspects, the at least two supply electrodes can be separated by a distance along a longitudinal axis of the lead body.

In a thirty-second aspect, in addition to one or more of the preceding or following aspects, or in the alternative to some aspects, the implantable lead can further include a temperature sensor, wherein the temperature sensor can be disposed between the at least two supply electrodes.

In a thirty-third aspect, an implantable lead for a cancer treatment system can be included having a lead body including a proximal end and a distal end and one or more supply electrodes, wherein the electrodes can be disposed along a length of the lead body. The implantable lead can also include a coating, wherein the coating can be formed of an expandable material. The coating can be disposed over at least one of the electrodes and/or a location adjacent the electrodes.

In a thirty-fourth aspect, in addition to one or more of the preceding or following aspects, or in the alternative to some aspects, the electrodes can be disposed circumferentially around the lead body and extend a distance along a longitudinal axis of the lead body.

In a thirty-fifth aspect, in addition to one or more of the preceding or following aspects, or in the alternative to some aspects, the electrodes can include a flat ribbon of metal.

In a thirty-sixth aspect, in addition to one or more of the preceding or following aspects, or in the alternative to some aspects, the flat ribbon of metal can be wrapped around the lead body.

In a thirty-seventh aspect, in addition to one or more of the preceding or following aspects, or in the alternative to some aspects, the electrodes can include at least two supply electrodes.

In a thirty-eighth aspect, in addition to one or more of the preceding or following aspects, or in the alternative to some aspects, the at least two supply electrodes can be separated by a distance along a longitudinal axis of the lead body.

In a thirty-ninth aspect, in addition to one or more of the preceding or following aspects, or in the alternative to some aspects, the implantable lead can further include a temperature sensor, wherein the temperature sensor can be disposed between the at least two supply electrodes.

In a fortieth aspect, in addition to one or more of the preceding or following aspects, or in the alternative to some aspects, the expandable material can be configured to swell in an aqueous environment.

In a forty-first aspect, in addition to one or more of the preceding or following aspects, or in the alternative to some aspects, the expandable material can be configured to swell such that a thickness thereof increases by 1 to 6 times.

In a forty-second aspect, in addition to one or more of the preceding or following aspects, or in the alternative to some aspects, the expandable material includes at least one of PMMA and PVA.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following drawings, in which:

FIG. 1 is a schematic view of a medical device implanted in a patient in accordance with various embodiments herein.

FIG. 2 is a schematic view of a placement of various cancer therapy leads in a region of a tumor resection site in accordance with various embodiments herein.

FIG. 3 is a schematic view of a cancer therapy lead in accordance with various embodiments herein.

FIG. 4 is a cross-sectional schematic view of a cancer therapy lead in accordance with various embodiments herein.

FIG. 5 is a schematic illustration of a portion of a cancer therapy lead interfacing with tissue of a patient in accordance with various embodiments herein.

FIG. 6 is a schematic view of a portion of a cancer therapy lead in accordance with various embodiments herein.

FIG. 7 is a schematic view of a portion of a cancer therapy lead in accordance with various embodiments herein.

FIG. 8 is a schematic view of a portion of a cancer therapy lead in accordance with various embodiments herein.

FIG. 9 is a schematic view of a portion of a cancer therapy lead in accordance with various embodiments herein.

FIG. 10 is a schematic view of a portion of a cancer therapy lead in accordance with various embodiments herein.

FIG. 11 is a simplified schematic view of a tapered portion of an electrode in accordance with various embodiments herein.

FIG. 12 is a simplified schematic view of a tapered portion of an electrode in accordance with various other embodiments herein.

FIG. 13 is a schematic view of a cancer therapy lead in accordance with various embodiments herein.

FIG. 14 is a schematic view of a cancer therapy lead in accordance with various embodiments herein.

FIG. 15 is a schematic view of a cancer therapy lead in accordance with various embodiments herein.

FIG. 16 is a schematic view of a cancer therapy lead in accordance with various embodiments herein.

FIG. 17 is a schematic cross-sectional view of a portion of a cancer therapy lead in accordance with various embodiments herein.

FIG. 18 is a schematic cross-sectional view of a medical device in accordance with various embodiments herein.

FIG. 19 is a schematic diagram of components of a medical device in accordance with various embodiments herein.

While embodiments are susceptible to various modifications and alternative forms, specifics thereof have been shown by way of example and drawings, and will be described in detail. It should be understood, however, that the scope herein is not limited to the particular embodiments described. On the contrary, the intention is to cover modifications, equivalents, and alternatives falling within the spirit and scope herein.

### DETAILED DESCRIPTION

As described above, various cancer therapies may have side effects on heathy tissue. Such side effects can be significant and thus it is advantageous to minimize them as much as possible.

Cancer therapies including the application of electrical fields may generate an amount of waste heat. For example, generation of electrical fields to prevent and/or disrupt cellular mitosis may generate heat. While the amount of heat generated for electrical field therapy to prevent and/or disrupt cellular mitosis may be substantially less than with other techniques such as electrical stimulation for tissue ablation, the heat may be enough to raise the temperature of tissues to levels resulting in negative tissue effects and/or tissue damage. Issues with heat exposure can be particularly important in the context of treating tissue inside the head such as in the case of a brain tumor and/or the treatment of a tumor resection site.

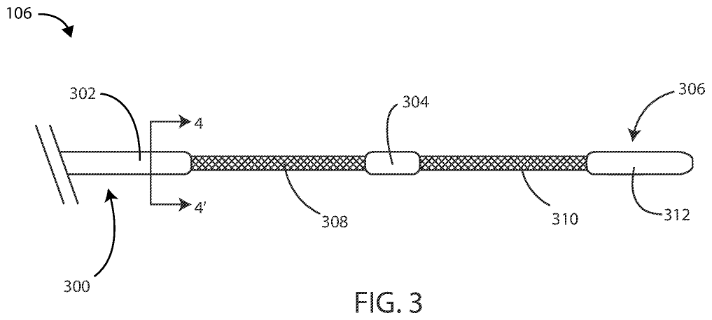

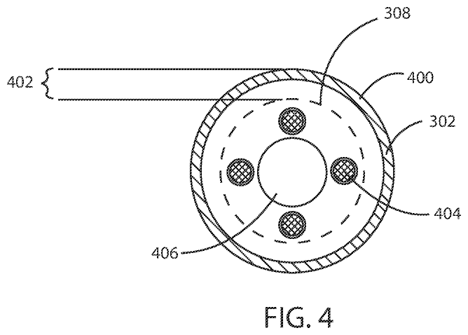

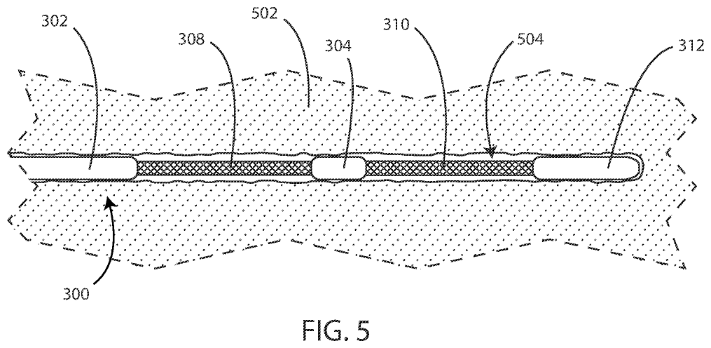

In some embodiments of systems herein electrodes are configured to result in a gap (or tissue exclusion zone or channel) between the surface of the electrode and the adjacent tissue into which the electrode is introduced. While not intending to be bound by theory, after placement into tissue intracranially, the gap space is filled by fluid which can aid in dissipating and/or dispersing heat generated by the electrodes thereby reducing the potential for heat damage to the adjacent tissue. The gap (or tissue exclusion zone or channel) can result from various physical configurations of leads and/or electrodes herein. In some embodiments, the outer diameter of an electrode can be smaller than the outer diameter of an adjoining portion of the lead body (see, e.g., FIGS. 3 and 4 herein). This difference in diameters creates a gap space after the lead is inserted into tissue of a patient (see, e.g., FIG. 5 herein). It will be appreciated that other structures can also be used to create the gap space (or tissue exclusion zone or channel) beyond just a difference in diameters of the electrode and an adjoining portion of the lead body and are contemplated herein.

In some embodiments, electrodes can be configured such that they are tapered near their boundary with adjoining portions of the lead body. While not intending to be bound by theory, it is believed that such a taper can reduce edge effects that may otherwise serve to generate and/or concentrate heat in such area. These features and others will now be described in greater detail.

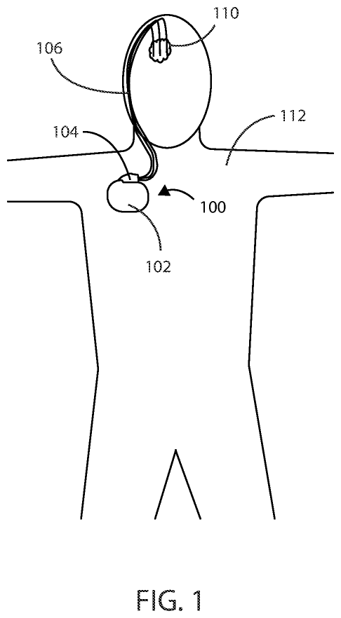

Referring now to FIG. 1, a schematic view of a medical device 100 implanted in a patient 112 is shown in accordance with the embodiments herein. In FIG. 1, the patient 112 has the medical device 100 implanted entirely within the body of the patient 112 at or near a tumor resection site 110. It will be appreciated that while many embodiments herein disclose a tumor resection site 110, area 110 may alternatively represent a cancerous or non-cancerous tumor site, zone, or cavity. Various implant sites can be used including areas such as in the limbs, the upper torso, the abdominal area, the head, and the like. In some embodiments, the medical device can be at least partially implanted within the body of the patient at or near the site of the cancerous tumor.

The medical device 100 can include a housing 102 and a header 104 coupled to the housing 102. Various materials can be used. However, in some embodiments, the housing 102 can be formed of a material such as a metal, ceramic, polymer, composite, or the like. In some embodiments, the housing 102, or one or more portions thereof, can be formed of titanium. The header 104 can be formed of various materials, but in some embodiments the header 104 can be formed of a translucent polymer such as an epoxy material. In some embodiments the header 104 can be hollow. In other embodiments the header 104 can be filled with components and/or structural materials such as epoxy or another material such that it is non-hollow.

In some embodiments where a portion of the medical device 100 is partially external, the header 104 and housing 102 can be surrounded by a protective casing made of durable polymeric material. In other embodiments, where a portion of the medical device 100 is partially external, the header 104 and housing 102 can be surrounded by a protective casing made of a combination of polymeric material, metallic material, and/or glass material.

Header 104 can be coupled to one or more leads, such as leads 106. The header 104 can serve to provide fixation of the proximal end of one or more leads 106 and electrically couple the one or more leads 106 to one or more components within the housing 102.

The one or more leads 106 can include one or more electrodes (not shown in this view) disposed along the length of the leads 106. In some embodiments, electrodes can include supply electrodes, also referred to herein as “electric field therapy supply electrodes.” In some embodiments electrodes can include electric field sensing electrodes, also referred to herein as “sensing electrodes.” In some embodiments, leads 106 can include both supply electrodes and sensing electrodes. In other embodiments, leads 106 can include any number of electrodes that are both supply electrodes and sensing electrodes.

The one or more leads 106 can also include one or more temperature sensors (not shown in this view) disposed along the length of the leads 106. Temperature sensors herein can include, but are not limited to, various types of optical and electrical temperature sensors. Temperature sensors herein can include contact-type temperature sensors and non-contact type temperature sensors. Optical temperature sensors herein can include infrared optical temperature sensors. Some optical temperature sensors can measure temperature at a distance such as a distance of millimeters or centimeters. Thus, even where temperature sensors are mounted along a lead 106, temperature can be measured at a distance therefrom. Exemplary electrical temperature sensors can include, but are not limited to, thermistors, resistive temperature detectors, thermocouples, semiconductor based temperature sensors, and the like.

In some embodiments, the medical device system can include a temperature sensor disposed remotely from the medical device. A remote temperature sensor can provide temperature data in addition to or in replace of temperature sensors in other areas such as along the leads 106. In some embodiments, a remote temperature sensor can be used to gather a core or reference temperature of the patient into which the system is implanted.

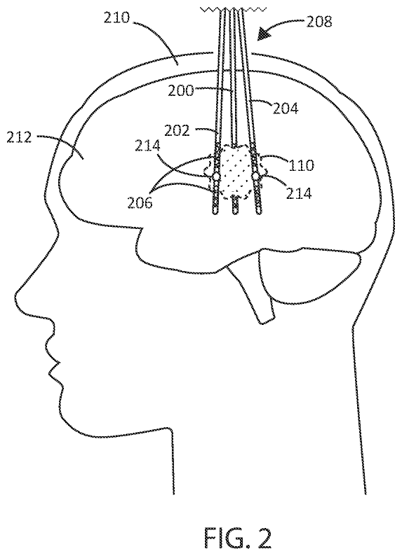

In some embodiments, the medical device can include a plurality of therapy leads implanted at or near a site a cancerous tumor or tumor resection. Referring now to FIG. 2, a schematic view of a placement of various cancer therapy leads 200, 202, 204 in a region of a tumor resection site 110 or tumor is shown in accordance with various embodiments herein. In the example of FIG. 2, temperature sensors 214 are disposed on the therapy leads. The temperature sensors 214 can be of any type described elsewhere herein. In the embodiment shown in FIG. 2, each cancer therapy lead includes two supply electrodes 206 disposed along a length of the cancer therapy leads. Each cancer therapy lead includes a proximal and a distal supply electrode. The cancer therapy leads and the supply electrodes disposed thereon are discussed in more detail below.

The side view shown in FIG. 2 also includes the placement of cancer therapy leads 200, 202, and 204 around the tumor resection site 110 and in position within a burr hole 208 entry point on the patient's skull 210 within the patient's brain 212. It will be appreciated that in some embodiments one burr hole can be used with one or more (e.g., one, two, three, or more) leads and/or electrodes. In some embodiments, multiple burr holes can be used each with one or more (e.g., one, two, three, or more) leads and/or electrodes.

Referring now to FIG. 3, a schematic view of an exemplary cancer therapy lead 106 is shown in accordance with various embodiments herein. The cancer therapy lead 106 can include a lead body 300 including lead body portion 302 (or proximal side portion) with a distal end 306. In this example, a first electrode 308 and a second electrode 310 are coupled to the lead body 300, such as positioned near a distal end 306 thereof. The electrodes 308, 310 can be supply electrodes. The electrodes 308, 310 can be electrically connected or electrically isolated from one another. The lead body 300 can include an interior portion and, in some embodiments, can define a lumen. The electrodes 308, 310 can include various conductive materials such as platinum, silver, gold, iridium, titanium, and various alloys. In some embodiments, the cancer therapy lead 106 includes more than two electrodes.

In some embodiments, the cancer therapy lead 106 can include one or more therapy zone temperature sensors disposed along a length of the cancer therapy lead. For example, a therapy zone temperature sensor can be positioned between the first electrode 308 and the second electrode 310. The therapy zone temperature sensor can include an optical or electrical thermal sensor. For example, the therapy zone temperature sensor can include a thermistor. The therapy zone temperature sensor can be used to measure the thermal heating about the cancer therapy lead to provide feedback to a clinician about the local thermal heating zone around the lead and provide a tissue temperature of the treatment site to the medical device. In various embodiments, the therapy zone temperature sensor can provide a tissue temperature at a site offset from a surface of the electrodes 308, 310. If a tissue temperature of a site offset from the electrodes 308, 310 is measured, the medical device can compensate for the offset when measuring or estimating the temperature of the tissue.

In some embodiments, the cancer therapy lead 106 can further include an end portion 312. In some embodiments, the end portion 312 can include a fixation element, such as an element that can adhere to a portion of the subject's body to maintain the position of the cancer therapy lead 106 and/or the electrodes 308. In various embodiments, the end portion 312 can be disposed at the distal end 306 of the cancer therapy lead 106.

In various embodiments, the diameter of the electrodes 308, 310 can be less than the diameter of the lead body portion 302, at least where the lead body portion 302 borders the electrodes. In this manner, a gap space (or tissue exclusion zone or channel) is created around the electrodes after the lead is inserted into tissue of a patient (see, e.g. FIG. 5 herein). As such, the lead body portion 302, spacing portion 304, and end portion 312 can define sides of one or more tissue exclusion channels. It will be appreciated, however, that other structures can also be used to create the gap space.

Referring now to FIG. 4, a cross-sectional schematic view of a cancer therapy lead as taken along line 4-4′ of FIG. 3 is shown in accordance with various embodiments herein. The cancer therapy lead can include an outer layer 400 with an outer surface. The outer layer 400 can be flexible and can be configured to protect other components disposed within the lumen of the outer layer 400. In some embodiments, the outer layer 400 can be circular in cross-section. In some embodiments, the outer layer 400 includes a dielectric material and/or an insulator. In some embodiments, the outer layer 400 can include various biocompatible materials such as polysiloxanes, polyethylenes, polyamides, polyurethane and the like.

In various embodiments, the cancer therapy lead 106 can include one or more conductors (wires, cables, etc.), such as a first conductor 404 and a second conductor 406 disposed therein. In some embodiments, the first conductor 404 and the second conductor 406 can be disposed within the lumen of the outer layer 400. The first conductor 404 and a second conductor 406 can be configured to provide electrical communication between an electrode 308 and the proximal end of the cancer therapy lead 106. The first conductor 404 and a second conductor 406 can include various materials including copper, aluminum, silver, gold, and various alloys such as tantalum/platinum, MP35N and the like. An insulator 408 and 410 can surround the first conductor 404 and a second conductor 406. The insulators 408 and 410 can include various materials such as electrically insulating polymers.

In some embodiments, each of the electrodes 308 can have individual first conductors 404 and second conductors 406 to electrically couple the electrode 308 to the proximal end of the cancer therapy lead 106. However, in some embodiments, each of the electrodes 308 only connects to a single conductor to electrically couple the electrode 308 to the proximal end of the cancer therapy lead 106. In some embodiments, the first conductor 404 and a second conductor 406 can be configured as a coil or a cable. Multiple conductors can be disposed within the lumen of the outer layer 400. For example, a separate conductor or set of conductors can be in communication with each electrode disposed along the lead. In various embodiments, a first conductor 404 and a second conductor 406 can form a part of an electrical circuit by which the electric fields from the electric field generating circuit are delivered to the site of the cancerous tissue.

In some embodiments, the cancer therapy lead 106 can include a central channel 412. The central channel 412 can be configured for a guide wire, or other implanting device, to pass through, such as to aid in implanting the cancer therapy lead 106 and electrodes 308. In some cases, additional channels (not shown) are disposed within the cancer therapy lead 106.

In the example of FIG. 4, the outside circumference of the electrode 308 can be seen. The outside circumference of the lead body portion 302 can also be seen. The outside circumference of electrode 308 has a smaller diameter than that of lead body portion 302. In some embodiments, the spacing portion 304 and end portion 312 can have the same or similar outside circumference as the lead body portion 302. The distance 402 between the respective outside surfaces (or gap distance) can be about 0.1, 0.2, 0.5, 1, 2, 3, 4, 5, or 7.5 mm, or a distance falling within a range between any of the foregoing. In some embodiments, the gap distance 402 can be substantially consistent around the circumference of the lead. However, in some embodiments, the gap distance 402 can be asymmetrical around the circumference being greater at some radial positions around the lead versus other.

As referenced above, a gap space is present after the lead is inserted into tissue of a patient. The gap space can fill up with fluid after placement of the lead. Referring now to FIG. 5, a schematic illustration is shown of a portion of a cancer therapy lead interfacing with tissue 502 of a patient in accordance with various embodiments herein. As before, the cancer therapy lead includes a lead body 300, with a lead body portion 302, first electrode 308, spacing portion 304, second electrode 310, end portion 312, and distal end 306. Gap space 504 (or tissue exclusion channel) surrounds the first electrode 308 and the second electrode 310 in this example. Gap space 504 can be filled with a fluid after placement of the lead. For example, after placement of the lead intracranially gap space 504 can be filled with cerebrospinal fluid (CSF). While not intending to be bound by theory, the fluid can aid in dissipating and/or dispersing heat generated by the electrodes thereby reducing the potential for heat damage to the adjacent tissue, such as adjacent neural tissue. In some embodiments, a rapidly dissolving composition can be packed within the tissue exclusion channel. The rapidly dissolving composition can take up the space within the tissue exclusion channel initially (such as before and during insertion of the electrical therapy lead) but dissolve after the lead is placed inside the body. In some embodiments, the rapidly dissolving composition can be a biocompatible composition such as a biocompatible carbohydrate composition, a biocompatible salt composition, or the like.

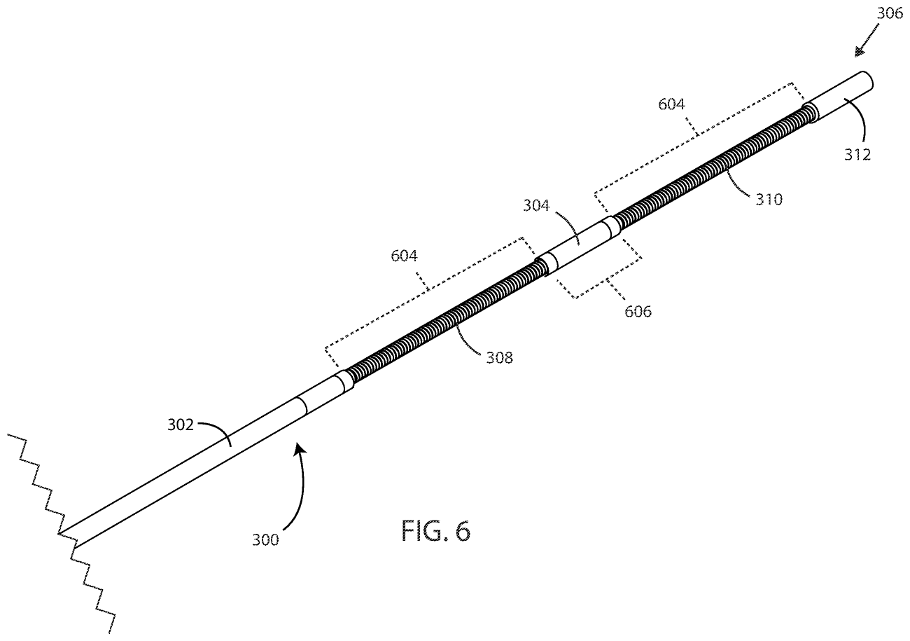

Referring now to FIG. 6, a schematic perspective view is shown of a portion of a cancer therapy lead in accordance with various embodiments herein. As before, the lead includes electrodes 308, 310 disposed along a length of the lead body 300, where the electrodes are separated by a spacing portion 304 on the exterior of the lead. In this view, the lead also is shown to include end portion 312 and distal end 306. The electrodes can include an electrode length 604, and the non-conducting portion can include a spacing portion length 606.

The electrode lengths 604 can each independently be from 1 cm to 4 cm in length. In some embodiments, the electrode length can be greater than or equal to 1.0 cm, 1.5 cm, 2.0 cm, 2.5 cm, 3.0 cm, 3.5 cm, 4.0 cm, 4.5 cm, 5.0 cm, 5.5 cm, or 6.0 cm, or can be an amount falling within a range between any of the foregoing. In various embodiments, when more than one electrode is present along a length of the lead body, each electrode can be the same length. In various other embodiments, when more than one electrode is present along a length of the lead body, each electrode can be a different length.

For leads including two or more electrodes, the spacing portion length 606 can be from 0.5 cm to 2 cm. In some embodiments, the non-conducing gap portion length can be greater than or equal to 0.25 cm, 0.50 cm, 0.75 cm, 1.00 cm, 1.25 cm, 1.50 cm, 1.75 cm, 2.00 cm, 2.25 cm, 2.5 cm, 2.75 cm, or 3 cm, or can be an amount falling within a range between any of the foregoing.

The gap space can be formed using various different physical structures. In some embodiments, the lead body can be a be a substantially similar diameter along its length. In other embodiments, the portion of the lead body adjoining the electrodes may have a larger diameter so as to create a gap space. As an example, the portion of the lead body adjoining the electrodes can include a spacing structure such as a shoulder structure or a spacing flange that results in a difference between the outer diameter of the electrode and the outer diameter of an adjoining portion of the lead body.

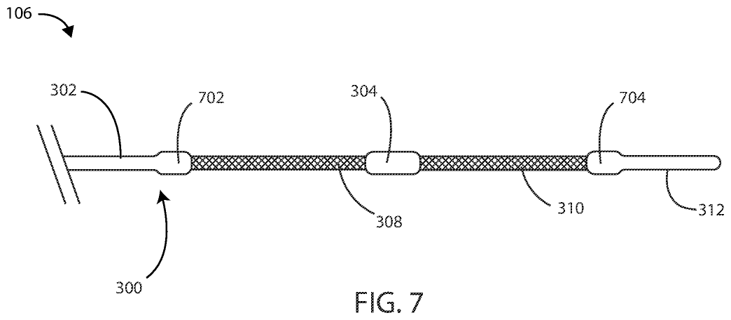

Referring now to FIG. 7, a schematic view is shown of a portion of a cancer therapy lead 106 in accordance with various embodiments herein. As before, the cancer therapy lead 106 includes lead body 300 including a lead body portion 302, first electrode 308, spacing portion 304, second electrode 310, end portion 312, and distal end 306.

In this example, the lead also includes first spacing structure 702 (or spacing ridge) and a second spacing structure 704 (or spacing ridge). In this specific example, the first spacing structure 702 is positioned on the edge of the lead body portion 302 adjoining the proximal end of the first electrode 308 and the second spacing structure 704 is positioned on the edge of the lead body portion 302 adjoining the distal end of the second electrode 310. In some embodiments, the spacing structures 702 are integral with lead body portion 302. In other embodiments, the spacing structure 702 may be structural separate components fitted over and/or against the lead body portion 302.

In some embodiments, the gap space may be created solely by structural features near the proximal or distal ends of the electrodes. However, in other embodiments various structures can be placed over the electrodes themselves in order to create and/or assist in maintaining a gap space.

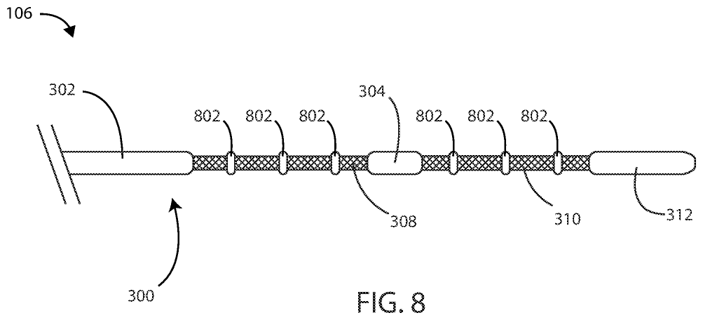

Referring now to FIG. 8, a schematic view is shown of a portion of a cancer therapy lead 106 in accordance with various embodiments herein. As before, the cancer therapy lead 106 includes a lead body 300, including a lead body portion 302, first electrode 308, spacing portion 304, second electrode 310, end portion 312, and distal end 306. In this example, the lead 106 also includes spacer rings 802 (or support flanges or in some cases spacing ridges) disposed over the electrodes 308, 310. The spacer rings 802 can function to maintain a gap space and prevent tissue from contacting the surface of the electrodes after the cancer therapy lead 106 is inserted into tissue of a patient. The spacer rings 802 can have an outside diameter that is greater than the electrodes they are placed over creating the gap space. For example, the spacer rings 802 can have an outside diameter that is greater than the electrodes by about 0.1, 0.2, 0.5, 1, 2, 3, or 4 mm, or a distance falling within a range between any of the foregoing. In some embodiments, the spacer rings can be formed of a polymer, but other materials can also be used. FIG. 8 shows three spacer rings 802 per electrode, however it will be appreciated that various number of spacer rings 802 per electrode are contemplated herein such as 1, 2, 3, 4, 5, 6, 7 or more, or an amount falling within a range between any of the foregoing.

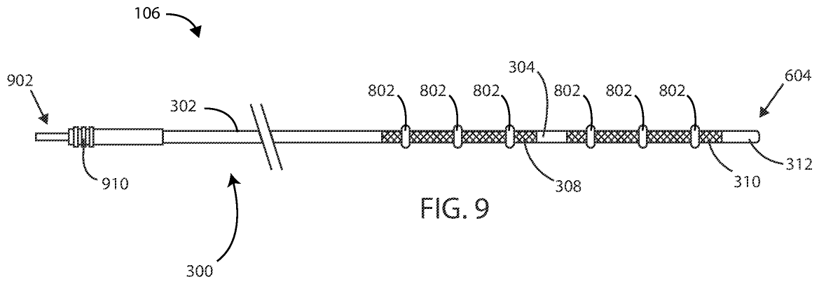

In the example of FIG. 8, the spacer rings can work to form and/or maintain the gap space in combination with a difference in outer diameter of the electrodes versus adjoining portions of the lead body. However, in other embodiments, spacer rings can form and/or maintain the gap space on their own. Referring now to FIG. 9, a schematic view is shown of a portion of a cancer therapy lead in accordance with various embodiments herein. As before, the cancer therapy lead 106 includes a lead body 300, a lead body portion 302, first electrode 308, spacing portion 304, second electrode 310, end portion 312, and distal end 306. The view of the cancer therapy lead 106 in FIG. 9 also shows the proximal end 902 of the lead along with a terminal pin 910 or plug for connecting the cancer therapy lead 106 to a medical device, such as a cancer treatment device. The terminal pin 910 or plug can be compatible with various standards for lead-header interface design including the DF-1, VS-1, IS-1, LV-1 and IS-4 standards, amongst other standards.

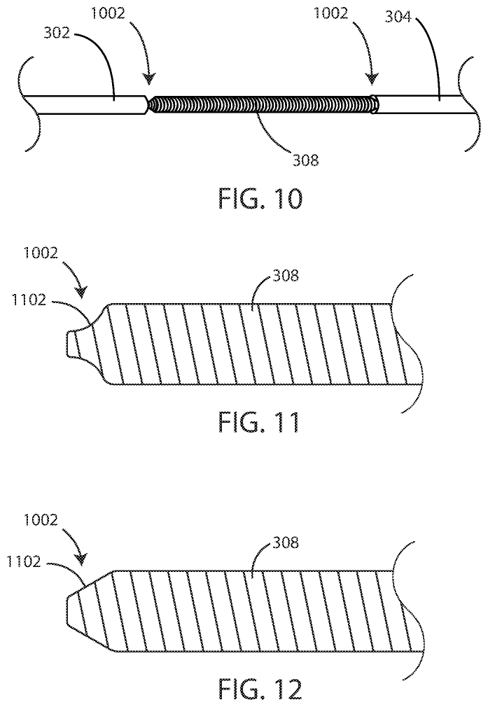

In some embodiments, electrodes herein can be configured such that they are tapered inward near their boundary with adjoining portions of the lead body. While not intending to be bound by theory, it is believed that such a taper can reduce edge effects that may otherwise serve to generate and/or concentrate heat in such area. Referring now to FIG. 10, a schematic view is shown of a portion of a cancer therapy lead in accordance with various embodiments herein. In this view, the cancer therapy lead is shown to include lead body portion 302, first electrode 308, and spacing portion 304. In this example, the edges 1002 of the electrode 308 are tapered such that the edges 1002 have a smaller outside diameter than other portions of the electrode 308, such as a middle portion of the electrode 308. For example, a diameter at the proximal edge of the electrode is at least 10% less than a diameter at a midpoint between the proximal edge and the distal edge. Similarly, a diameter at the distal edge of the electrode is at least 10% less than a diameter at a midpoint between the proximal edge and the distal edge.

In some embodiments, the tapered portion can be curved and in other embodiment the tapered portion can be substantially straight. In some embodiments, the tapered portion can be concave and in other embodiments convex. Referring now to FIG. 11, a simplified schematic view is shown of a tapered portion of an electrode 308 in accordance with various embodiments herein. In this example, the tapered portion 1102 of the edge 1002 of the electrode 308 can include a concave curvature. Referring now to FIG. 12, a simplified schematic view is shown of a tapered portion of an electrode 308 in accordance with various other embodiments herein. In this example, the tapered portion 1102 of the edge 1002 of the electrode 308 can include a substantially straight portion.

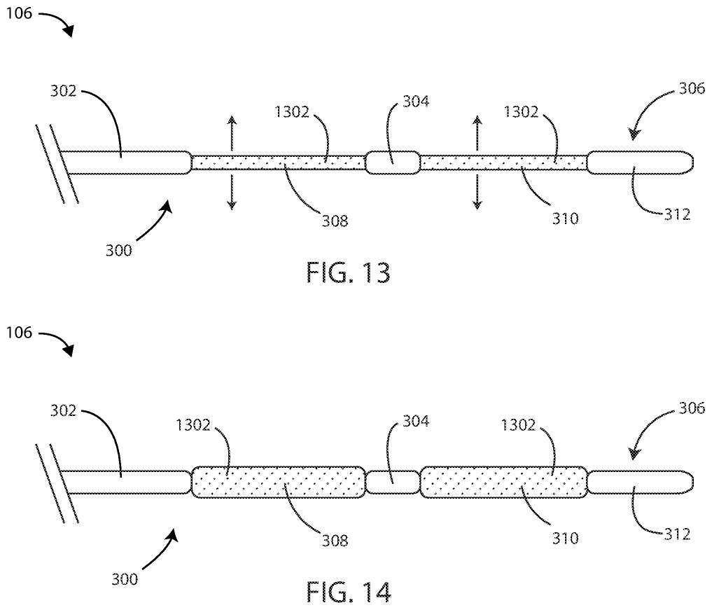

In some embodiments, one or more portions of the lead can be covered with a coating of an expandable material. For example, in some cases electrodes can be covered with a coating of an expandable porous material. Referring now to FIG. 13, a schematic view of a cancer therapy lead 106 is shown in accordance with various embodiments herein. In this example, the cancer therapy lead 106 can include a lead body 300, a lead body portion 302, and a distal end 306. In some embodiments, the cancer therapy lead 106 can further include an end portion 312. The end portion 312 can be disposed at the distal end 306 of the cancer therapy lead 106. In this example, a first electrode 308 and a second electrode 310 are coupled to the lead body portion 302, such as positioned near a distal end 306 thereof. The electrodes 308, 310 can be supply electrodes.

The electrodes 308, 310 can be covered with a coating 1302 of an expandable material. The coating 1302 can wrap around the electrodes 308, 310 to form a sleeve-like structure. In various embodiments, to start, the diameter of the electrodes 308, 310 with the coating 1302 can be less than the diameter of the lead body portion 302, at least where the lead body portion 302 borders the electrodes. However, after insertion into a patient, the coating 1302 can expand. Referring now to FIG. 14, a schematic view of a cancer therapy lead 106 is shown in accordance with various embodiments herein. In this example, the cancer therapy lead 106 includes the same elements as described with respect to FIG. 13, however the coating 1302 has expanded outward. In this expanded state, the outer diameter of the coating 1302 is greater than the diameter of the lead body portion 302, at least where the lead body portion 302 borders the electrodes.

The coating can be formed of various materials and have various properties. In some embodiments, the coating is formed of an expandable (or swellable) porous material. In some embodiments, the material can be a hygroscopic coating that absorbs moisture from the in vivo environment to swell. In some embodiments, the material has a relatively slow absorption rate, but when fully saturated (fully expanded) results in an overall diameter equaling or exceeding that of around lead body portion 302, spacing portion 304, end portion 312.

In some embodiments, the moisture absorption rate can be such that it takes hours (e.g., at least 1, 2, 3, 6, or more hours) to saturation time (or full expansion). This can allow for implant position and reposition.

The coating can be configured such that there is a predictable final diameter after expansion. The full degree of expansion can be controlled based on the specific material used, the amount of deposited, the cross-link density or the material, as well as other parameters. In some embodiments, the thickness of the coating can be relatively thin before implantation and exposure to an aqueous environment. In some embodiments, the thickness of the coating prior to implantation can be about 0.05 to 1 mm or about 0.1 to 0.3 mm. In some embodiments, expansion (swelling) can increase the thickness at least about 0.5, 1, 2, 3, 4, 5, 6, 8, or 10 times or more, or an amount falling within a range between any of the foregoing.

In some embodiments, the polymer material of the coating can be a hydrogel. In many embodiments, the polymer or polymers can be a durable, non-degradable polymer permanently displacing tissue at the site of implantation. However, in some embodiments degradable polymers (such as those including hydrolytically labile bonds under physiological conditions) are also contemplated herein. In some embodiments, the material can be polymethylmethacrylate (PMMA), such as low crosslink density PMMA. Other materials can include, but are not limited to, polyvinyl alcohol (PVA), polyacrylamide, chitosan, polyvinyl pyrrolidone (PVP), alginates, hyaluronic acid, polyethylene glycol, cellulose acetate, and the like.

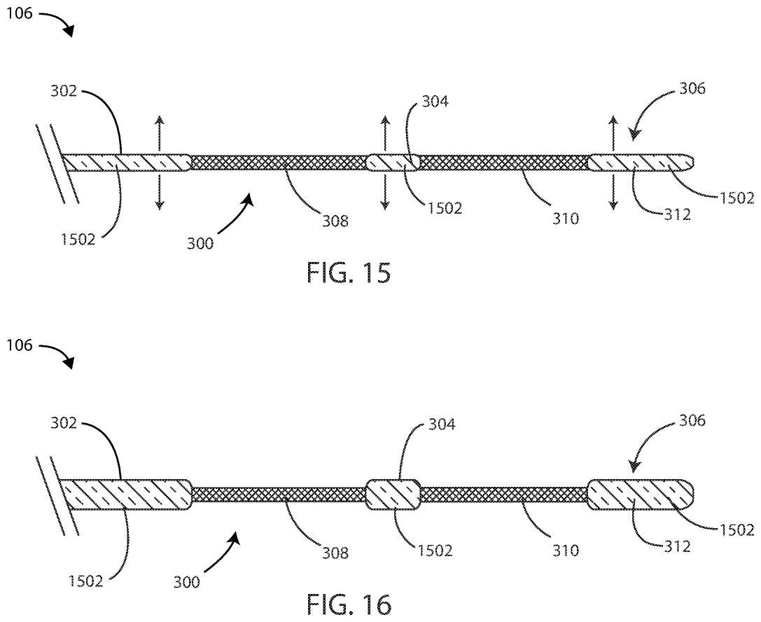

Other embodiments herein can also include one or more expandable portions. For example, one or more of the lead body portion 302, spacing portion 304, end portion 312 can include a coating of an expandable material thereon. Referring now to FIG. 15, a schematic view of a cancer therapy lead 106 is shown in accordance with various embodiments herein. In this example, the cancer therapy lead 106 can include a lead body 300 with a lead body portion 302 with a distal end 306. In some embodiments, the cancer therapy lead 106 can further include an end portion 312. The end portion 312 can be disposed at the distal end 306 of the cancer therapy lead 106. In this example, a first electrode 308 and a second electrode 310 are coupled to the lead body portion 302, such as positioned near a distal end 306 thereof. The electrodes 308, 310 can be supply electrodes.

In this example, lead body portion 302, spacing portion 304, end portion 312 can be covered with a coating 1502 of an expandable material. The coating 1502 can wrap around lead body portion 302, spacing portion 304, end portion 312 to form a sleeve-like structure. However, after insertion into a patient, the coating 1502 can expand. Referring now to FIG. 16, a schematic view of a cancer therapy lead 106 is shown in accordance with various embodiments herein. In this example, the cancer therapy lead 106 includes the same elements as described with respect to FIG. 15x, however the coating 1502 has expanded outward. Such expansion can generate a gap space (or tissue exclusion zone or channel) around the electrodes after the lead is inserted into tissue of a patient (such as described with respect to other embodiments herein). In some embodiments, the material for the coating 1502 can be the same as or different than coating 1302 described with respect to FIGS. 13 and 14.

In some embodiments, various other parts of cancer therapy leads can be formed with expandable materials including, but not limited to, spacing ridges and/or flanges.

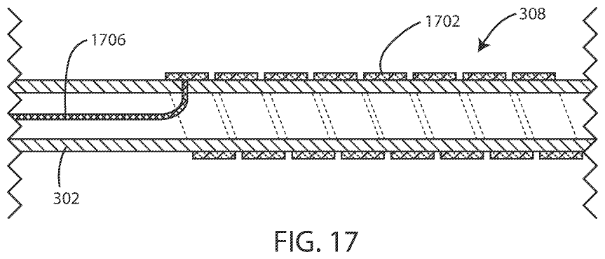

Referring now to FIG. 17, a schematic cross-sectional view is shown of a portion of a cancer therapy lead in accordance with various embodiments herein. As before, the cancer therapy lead includes lead body portion 302 and a first electrode 308. The electrode 308 can be formed of a plurality of conductive ribbon segments 1702 that can be in electrical communication with each other and with conductor 1706. The conductive ribbon segments 1702 can be disposed circumferentially about lead body portion 302 (such as wrapped around lead body portion 302 helically). In this example, the electrodes can be formed with a flat ribbon of metal. However, it will be appreciated that electrodes herein can take on various other forms and/or be formed with different materials including a wrapped wire, a tubular structure, a deposited metal layer, or the like.

### Medical Device Components

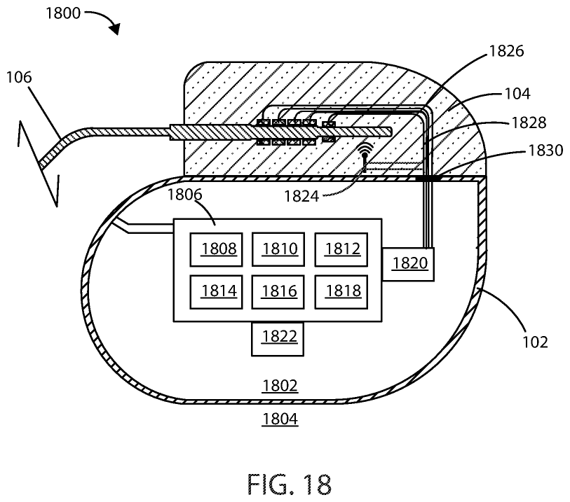

Referring now to FIG. 18, a schematic cross-sectional view of an exemplary medical device 1800 is shown in accordance with various embodiments herein. The housing 102 can define an interior volume 1802 that can be hollow and that in some embodiments is hermetically sealed off from the area 1804 outside of medical device 1800. In other embodiments the housing 102 can be filled with components and/or structural materials such that it is non-hollow. The medical device 1800 can include control circuitry 1806, which can include various components 1808, 1810, 1812, 1814, 1816, and 1818 disposed within housing 102. In some embodiments, these components can be integrated and in other embodiments these components can be separate. In yet other embodiments, there can be a combination of both integrated and separate components. The medical device 1800 can also include an antenna 1824, to allow for unidirectional or bidirectional wireless data communication, such as with an external device or an external power supply. In some embodiments, the components of medical device 1800 can include an inductive energy receiver coil (not shown) communicatively coupled or attached thereto to facilitate transcutaneous recharging of the medical device via recharging circuitry.

The various components 1808, 1810, 1812, 1814, 1816, and 1818 of control circuitry 1806 can include, but are not limited to, a microprocessor, memory circuit (such as random access memory (RAM) and/or read only memory (ROM)), recorder circuitry, controller circuit, a telemetry circuit, a power supply circuit (such as a battery), a timing circuit, and an application specific integrated circuit (ASIC), a recharging circuit, amongst others. Control circuitry 1806 can be in communication with an electric field generating circuit 1820 that can be configured to generate electric current to create one or more fields. The electric field generating circuit 1820 can be integrated with the control circuitry 1806 or can be a separate component from control circuitry 1806. Control circuitry 1806 can be configured to control delivery of electric current from the electric field generating circuit 1820. In some embodiments, the electric field generating circuit 1820 can be present in a portion of the medical device that is external to the body.

In some embodiments, the control circuitry 1806 can be configured to direct the electric field generating circuit 1820 to deliver an electric field via leads 106 to the site of a cancerous tumor located within a bodily tissue. In other embodiments, the control circuitry 1806 can be configured to direct the electric field generating circuit 1820 to deliver an electric field via the housing 102 of medical device 1800 to the site of a cancerous tumor located within a bodily tissue. In other embodiments, the control circuitry 1806 can be configured to direct the electric field generating circuit 1820 to deliver an electric field between leads 106 and the housing 102 of medical device 1800. In some embodiments, one or more leads 106 can be in electrical communication with the electric field generating circuit 1820.

In some embodiments, medical device 1800 can include an electric field sensing circuit 1822 configured to generate a signal corresponding to sensed electric fields. Electric field sensing circuit 1822 can be integrated with control circuitry 1806 or it can be separate from control circuitry 1806.

Sensing electrodes (not shown in this view) can be disposed on or adjacent to the housing of the medical device, on one or more leads connected to the housing, on a separate device implanted near or in the tumor, or any combination of these locations. In some embodiments, the electric field sensing circuit 1822 can measure the electrical potential difference (voltage) between a first electrode and a second electrode, wherein the first and second electrodes are in any of the aforementioned locations. In some embodiments, the electric field sensing circuit can be configured to measure sensed electric fields and to record electric field strength in V/cm.

In some embodiments, the one or more leads 106 can be in electrical communication with the electric field generating circuit 1820. The one or more leads 106 can include one or more electrodes. In some embodiments, various electrical conductors, such as electrical conductors 1826 and 1828, can pass from the header 104 through a feed-through structure 1830 and into the interior volume 1802 of medical device 1800. As such, the electrical conductors 1826 and 1828 can serve to provide electrical communication between the one or more leads 106 and control circuitry 1806 disposed within the interior volume 1802 of the housing 102.

In some embodiments, recorder circuitry can be configured to record the data produced by the electric field sensing circuit 1822 and record time stamps regarding the same. In some embodiments, the control circuitry 1806 can be hardwired to execute various functions, while in other embodiments the control circuitry 1806 can be directed to implement instructions executing on a microprocessor or other external computation device. A wireless communication interface can also be provided for communicating with external computation devices such as a programmer, a home-based unit, and/or a mobile unit (e.g., a cellular phone, personal computer, smart phone, tablet computer, smartwatch, and the like).

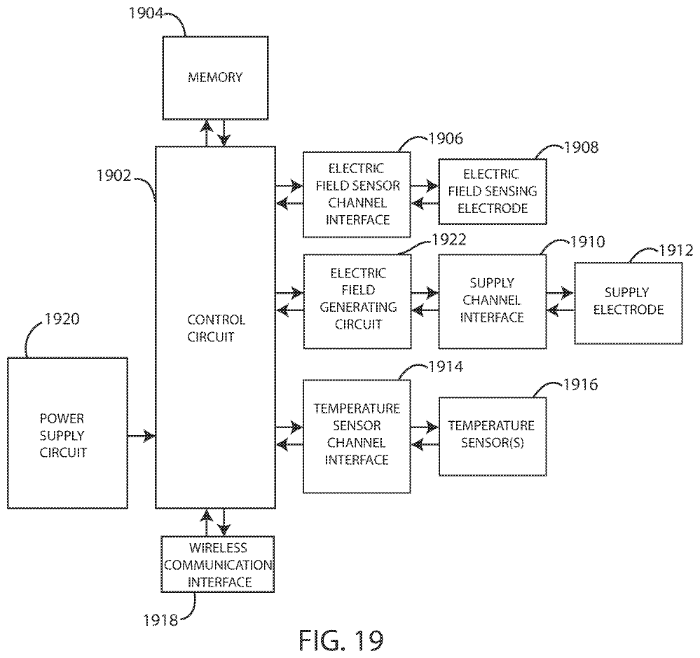

Elements of various embodiments of the medical devices described herein are shown in FIG. 19. However, it will be appreciated that some embodiments can include additional elements beyond those shown in FIG. 19. In addition, some embodiments may lack some elements shown in FIG. 19. The medical devices as embodied herein can gather information through one or more sensing channels and can output information through one or more field generating channels. A microprocessor 1902 can communicate with a memory 1904 via a bidirectional data bus. The memory 1904 can include read only memory (ROM) or random-access memory (RAM) for program storage and RAM for data storage. The microprocessor 1902 can also be connected to a wireless communication interface 1918 for communicating with external devices such as a programmer, a home-based unit and/or a mobile unit (e.g., a cellular phone, personal computer, smart phone, tablet computer, and the like) or directly to the cloud or another communication network as facilitated by a cellular or other data communication network. The medical device can include a power supply circuit 1920. In some embodiments, the medical device can include an inductive energy receiver coil interface (not shown) communicatively coupled or attached thereto to facilitate transcutaneous recharging of the medical device.

The medical device can include one or more electric field sensing electrodes 1908 and one or more electric field sensor channel interfaces 1906 that can communicate with a port of microprocessor 1902. The medical device can also include one or more electric field generating circuits 1922, one or more supply electrodes 1912, and one or more supply channel interfaces 1910 that can communicate with a port of microprocessor 1902. The medical device can also include one or more temperature sensors 1916 and one or more temperature sensor channel interfaces 1914 that can communicate with a port of microprocessor 1902. The channel interfaces 1906, 1910, and 1914 can include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like.

In some embodiments, one or more physiological sensors can also be included herein. In some embodiments, the physiological sensors can include sensors that monitor temperature, blood flow, blood pressure, and the like. In some embodiments, the respiration sensors can include sensors that monitor respiration rate, respiration peak amplitude, and the like. In some embodiments, the chemical sensors can measure the quantity of an analyte present in a treatment area about the sensor, including but not limited to analytes such as of blood urea nitrogen, creatinine, fibrin, fibrinogen, immunoglobulins, deoxyribonucleic acids, ribonucleic acids, potassium, sodium, chloride, calcium, magnesium, lithium, hydronium, hydrogen phosphate, bicarbonate, and the like. However, many other analytes are also contemplated herein. Exemplary chemical/analyte sensors are disclosed in commonly owned U.S. Pat. No. 7,809,441 to Kane et al., and which is hereby incorporated by reference in its entirety.

Although the temperature sensors 1916 are shown as part of a medical device in FIG. 19, it is realized that in some embodiments one or more of the sensors could be physically separate from the medical device. In various embodiments, one or more of the can be within another implanted medical device communicatively coupled to a medical device via wireless communication interface 1918. In yet other embodiments, one or more of the sensors can be external to the body and coupled to a medical device via wireless communication interface 1918.

### Electric Field Therapy Parameters

In some embodiments, medical devices herein can generate one or more electric fields at frequencies selected from a range of between 10 kHz to 1 MHz. In some embodiments, the one or more electric fields can be effective to prevent and/or disrupt cellular mitosis in a cell. In some embodiments, the one or more electric fields can be effective to prevent and/or disrupt cellular mitosis in a cell, but not cause tissue ablation. In some embodiments, the system can be configured to deliver an electric field at one or more frequencies selected from a range of within 300 kHz to 500 kHz. In some embodiments, the system can be configured to deliver an electric field at one or more frequencies selected from a range of within 100 kHz to 300 kHz. In some embodiments, the system can be configured to periodically deliver an electric field using one or more frequencies greater than 10 kHz.

A desired electric field strength can be achieved by delivering an electric current between two electrodes. The specific current and voltage at which the electric field is delivered can vary and can be adjusted to achieve the desired electric field strength at the site of the tissue to be treated. In some embodiments, the system can be configured to deliver an electric field using currents ranging from 1 mAmp to 1000 mAmp to the site of a cancerous tumor. In some embodiments, the system can be configured to deliver an electric field using currents ranging from 20 mAmp to 500 mAmp to the site of a cancerous tumor. In some embodiments, the system can be configured to deliver an electric field using currents ranging from 30 mAmp to 300 mAmp to the site of a cancerous tumor.

In some embodiments, the system can be configured to deliver an electric field using currents including 1 mAmp, 2 mAmp, 3 mAmp, 4 mAmp, 5 mAmp, 6 mAmp, 7 mAmp, 8 mAmp, 9 mAmp, 10 mAmp, 15 mAmp, 20 mAmp, 25 mAmp, 30 mAmp, 35 mAmp, 40 mAmp, 45 mAmp, 50 mAmp, 60 mAmp, 70 mAmp, 80 mAmp, 90 mAmp, 300 mAmp, 125 mAmp, 150 mAmp, 175 mAmp, 400 mAmp, 225 mAmp, 250 mAmp, 275 mAmp, 300 mAmp, 325 mAmp, 350 mAmp, 375 mAmp, 400 mAmp, 425 mAmp, 450 mAmp, 475 mAmp, 500 mAmp, 525 mAmp, 550 mAmp, 575 mAmp, 600 mAmp, 625 mAmp, 650 mAmp, 675 mAmp, 700 mAmp, 725 mAmp, 750 mAmp, 775 mAmp, 800 mAmp, 825 mAmp, 850 mAmp, 875 mAmp, 900 mAmp, 925 mAmp, 950 mAmp, 975 mAmp, or 1000 mAmp. It will be appreciated that the system can be configured to deliver an electric field at a current falling within a range, wherein any of the forgoing currents can serve as the lower or upper bound of the range, provided that the lower bound of the range is a value less than the upper bound of the range.

In some embodiments, the system can be configured to deliver an electric field using voltages ranging from 1 Vrms to 50 Vrms to the site of a cancerous tumor. In some embodiments, system can be configured to deliver an electric field using voltages ranging from 5 Vrms to 30 Vrms to the site of a cancerous tumor. In some embodiments, the system can be configured to deliver an electric field using voltages ranging from 10 Vrms to 20 Vrms to the site of a cancerous tumor.

In some embodiments, the system can be configured to deliver an electric field using one or more voltages including 1 Vrms, 2 Vrms, 3 Vrms, 4 Vrms, 5 Vrms, 6 Vrms, 7 Vrms, 8 Vrms, 9 Vrms, 10 Vrms, 15 Vrms, 20 Vrms, 25 Vrms, 30 Vrms, 35 Vrms, 40 Vrms, 45 Vrms, or 50 Vrms. It will be appreciated that the system can be configured to deliver an electric field at a voltage falling within a range, wherein any of the forgoing voltages can serve as the lower or upper bound of the range, provided that the lower bound of the range is a value less than the upper bound of the range.

In some embodiments, the system can be configured to deliver an electric field using one or more frequencies including 10 kHz, 20 kHz, 30 kHz, 40 kHz, 50 kHz, 60 kHz, 70 kHz, 80 kHz, 90 kHz, 300 kHz, 125 kHz, 150 kHz, 175 kHz, 400 kHz, 225 kHz, 250 kHz, 275 kHz, 300 kHz, 325 kHz, 350 kHz, 375 kHz, 400 kHz, 425 kHz, 450 kHz, 475 kHz, 500 kHz, 525 kHz, 550 kHz, 575 kHz, 600 kHz, 625 kHz, 650 kHz, 675 kHz, 700 kHz, 725 kHz, 750 kHz, 775 kHz, 800 kHz, 825 kHz, 850 kHz, 875 kHz, 900 kHz, 925 kHz, 950 kHz, 975 kHz, 1 MHz. It will be appreciated that the system can be configured to deliver an electric field using a frequency falling within a range, wherein any of the foregoing frequencies can serve as the upper or lower bound of the range, provided that the upper bound is greater than the lower bound.

In some embodiments, the system can be configured to generate one or more applied electric field strengths selected from a range of within 0.25 V/cm to 1000 V/cm, or 0.25 V/cm to 500 V/cm, or 0.25 V/cm to 100 V/cm, or 0.25 V/cm to 50 V/cm. In some embodiments, the system can be configured to generate one or more applied electric field strengths of greater than 3 V/cm. In some embodiments, the system can be configured to generate one or more applied electric field strengths selected from a range of within 1 V/cm to 10 V/cm. In some embodiments, the system can be configured to generate one or more applied electric field strengths selected from a range of within 3 V/cm to 5 V/cm.

In other embodiments, the system can be configured to deliver one or more applied electric field strengths including 0.25 V/cm, 0.5 V/cm, 0.75 V/cm, 1.0 V/cm, 2.0 V/cm, 3.0 V/cm, 5.0 V/cm, 6.0 V/cm, 7.0 V/cm, 8.0 V/cm, 9.0 V/cm, 10.0 V/cm, 20.0 V/cm, 30.0 V/cm, 40.0 V/cm, 50.0 V/cm, 60.0 V/cm, 70.0 V/cm, 80.0 V/cm, 90.0 V/cm, 300.0 V/cm, 125.0 V/cm, 150.0 V/cm, 175.0 V/cm, 400.0 V/cm, 225.0 V/cm, 250.0 V/cm, 275.0 V/cm, 300.0 V/cm, 325.0 V/cm, 350.0 V/cm, 375.0 V/cm, 400.0 V/cm, 425.0 V/cm, 450.0 V/cm, 475.0 V/cm, 500.0 V/cm, 600.0 V/cm, 700.0 V/cm, 800.0 V/cm, 900.0 V/cm, 1000.0 V/cm. It will be appreciated that the system can generate an electric field having a field strength at a treatment site falling within a range, wherein any of the foregoing field strengths can serve as the upper or lower bound of the range, provided that the upper bound is greater than the lower bound.

In some embodiments, an electric field can be applied to the site of a cancerous tumor or tumor resection at a specific frequency or constant frequency range.

In some embodiments, the electric field can be modulated in response to a patient's measured reference or core body temperature and/or a set period of time elapsing. For example, if the patient's reference or core body temperature is higher than a threshold level, the therapy parameters can be modulated to reduce the heat output of the system inside the body. If the patient's reference or core body temperature is higher than a threshold level, the electric field strength can be decreased by 5%, 10%, 15%, 20%, 25%, 30%, 35%, 40%, 45%, 50%, 55%, 60%, 65%, 70%, 75%, 80%, 85%, 90%, 95%, or 100% (e.g., turned off) or can be decreased by an amount falling within a range between any of the foregoing. If the patient's reference or core body temperature is higher than a threshold level, the electric field strength can be decreased by from 5% to 100%, or between 5% and 95%. It will be appreciated that other parameters can also be modulated in order to reduce the amount of heat generated by the system including, for example, current, voltage, and/or frequency.

Alternatively, if the reference or core body temperature is lower than a threshold level, then to maximize exposure to therapeutic electrical fields the electric field strength can be increased. In some embodiments, the electric field strength can be increased by 5%, 10%, 15%, 20%, 25%, 50%, 75%, 100%, 200%, 300%, 500%, 1000% or more, or by an amount falling within a range between any of the foregoing. It will be appreciated that other parameters can also be modulated in order to increase the intensity of the electrical field therapy provided by the system including, for example, current, voltage, and/or frequency.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An implantable lead for a cancer treatment system comprising:
a lead body, the lead body comprising
a proximal end;
a distal end; and
the lead body defining sides of a tissue exclusion channel;
one or more supply electrodes, wherein the electrodes are disposed along a length of the lead body; and
wherein the tissue exclusion channel is disposed circumferentially around the electrodes.

2. The implantable lead of claim 1, wherein the tissue exclusion channel has a radial depth of 0.1 to 4 mm and has an axial length of 1 to 200 mm.

3. The implantable lead of claim 1, further comprising one or more support flanges, wherein the one or more support flanges are disposed within the tissue exclusion channel.

4. The implantable lead of claim 1, further comprising a rapidly dissolving composition, wherein the rapidly dissolving composition is packed within the tissue exclusion channel.

5. The implantable lead of claim 1, the electrodes comprising a flat ribbon of metal.

6. The implantable lead of claim 5, wherein the flat ribbon of metal is wrapped around the lead body.

7. The implantable lead of claim 1, further comprising a coating;
wherein the coating is disposed over a surface of the supply electrodes; and
wherein the coating is formed of an expandable material.

8. The implantable lead of claim 1, further comprising a coating;
wherein the coating is disposed on the implantable lead at a location bordering the tissue exclusion channel; and
wherein the coating is formed of an expandable material.

9. An implantable lead for a cancer treatment system comprising:
a lead body, the lead body comprising
a proximal end; and
a distal end;
one or more supply electrodes, wherein the electrodes are disposed along a length of the lead body;
a spacing ridge;
wherein the spacing ridge is disposed on at least one of the lead body or the electrodes; and
wherein the spacing ridge extends radially from the implantable lead for a cancer treatment system and at least partially defines a tissue exclusion space adjacent the spacing ridge.

10. The implantable lead for a cancer treatment system of claim 9, further comprising a second spacing ridge, wherein the spacing ridge and the second spacing ridge are disposed on opposite sides of the electrodes.

11. The implantable lead for a cancer treatment system of claim 9, wherein the electrodes are disposed circumferentially around the lead body and extend a distance along a longitudinal axis of the lead body.

12. The implantable lead for a cancer treatment system of claim 9, the electrodes comprising a flat ribbon of metal.

13. The implantable lead for a cancer treatment system of claim 9, wherein the spacing ridge is formed of an expandable material.

14. An implantable lead for a cancer treatment system comprising:
a lead body, the lead body comprising
a proximal end; and
a distal end;
one or more supply electrodes, wherein the electrodes are disposed along a length of the lead body;
a coating;
wherein the coating is formed of an expandable material; and
wherein the coating is disposed over at least one of the electrodes and/or a location adjacent the electrodes.

15. The implantable lead of claim 14, wherein the electrodes are disposed circumferentially around the lead body and extend a distance along a longitudinal axis of the lead body.

16. The implantable lead of claim 14, the electrodes comprising a flat ribbon of metal.

17. The implantable lead of claim 16, wherein the flat ribbon of metal is wrapped around the lead body.

18. The implantable lead of claim 14, wherein the expandable material is configured to swell in an aqueous environment.

19. The implantable lead of claim 14, wherein the expandable material is configured to swell such that a thickness thereof increases by 1 to 6 times.

20. The implantable lead of claim 14, wherein the expandable material includes at least one of PMMA and PVA.
